(19) United States
(12) Patent Application Publication
(10) Pub. No.: US 2012/0149093 A1
(43) Pub. Date: Jun. 14, 2012

(54) Method and Apparatus for Automating Chemical and Biological Assays

(76) Inventors: Jason Gould, Mullica, NJ (US); Martin Gould, Mullica, NJ (US); Susan Gould, Mullica, NJ (US)

(21) Appl. No.: 13/287,306

(22) Filed: Nov. 2, 2011

Related U.S. Application Data
(63) Continuation-in-part of application No. 12/573,418, filed on Oct. 5, 2009.

(51) Int. Cl. C12M 1/40; G01N 33/48

(52) U.S. Cl. 435/286.2; 422/63

(57) ABSTRACT

A device which collects specimen fluids or performs chemical or biological assays of the specimen fluid is provided with a specimen fluid receiver and a fluid actuated expandable trigger coupled to receive specimen fluid from the specimen fluid receiver such that a predetermined delay occurs before the trigger expands sufficiently to move another component of the device, and the specimen fluid interacts with a substance during the delay. The trigger is made of a material which expands substantially upon absorbing specimen fluid, and it is mounted and positioned so as to contact and move the other component of the device upon expanding through the absorption of specimen fluid. The other component may contain a surface coupled to receive specimen fluid from the specimen fluid receiver and the surface has an area which contains a substance which interacts with the specimen fluid.

### RELATED APPLICATIONS

This application is a continuation-in-part of U.S. patent application Ser. No. 12/573,418, filed on Oct. 5, 2009, the entire disclosure of which is incorporated herein by reference.

### FIELD OF THE INVENTION

The present disclosure relates to the performance of chemical and biological assays and, more particularly, concerns a method and apparatus which permit the performance of complex, multistep assay procedures automatically, in a single operator-initiated process.

### BACKGROUND OF THE INVENTION

Monitoring and managing the public health depends very much upon the ability to perform chemical and biological assays, for example immunological assays, reliably and efficiently. In some instances, a health worker must obtain human or animal specimens in the field, usually with a handheld collection device. Such specimens may include, without limitation, urine, blood/plasma/senun, body fluids, synovial fluid, fecal matter, sweat, nasal aspirates, and tears. Once the specimen is taken, it must be retained safely and securely until it can be delivered to a central location. Often, it is desirable to add a substance to a specimen close to the time that it is taken. Most often, with devices that are to be inserted in the patient's mouth, such substances are added manually by an operator after the sample is taken, owing to the danger that substances which may be harmful may be communicated back to the patient (his mouth) through the collection device. On the other hand, it would be desirable for that substance to be contained in the collection device, both for convenience and to avoid any damage that may result from operator error.

Thus, there is a need for a collection device that can be isolated from the patient when a sample is taken, both for the security of the sample and to prevent communication back to the patient of substances contained in the collection device. Moreover, it is important that such isolation occurs automatically in order to prevent accidental damage to specimens or accidental injury to patients.

Chemical and biological assay devices and processes are known which accomplish complicated multistep processes in a single procedure. One example of such assays is “lateral flow” assays. However, it is often necessary or desirable to introduce a delay (an “incubation period”) after one step is performed and before the next one begins. Similarly, additives, for example, running buffer, may need to be introduced into a process after a certain delay. The operator must, for example, take a sample, add an additive, wait a prescribed amount of time, and then perform some other step. This demands diligence and skill on the part of the operator, not to mention rigorous training, as any inattention or error on his part can compromise the entire process. That is, waiting too long, or not long enough, can result in compromising the test results.

It would be desirable to have a multistep process involving delays between steps proceed automatically once it is initiated by an operator. This would not only improve the reliability and consistency of results, but it would allow the process to be performed by an operator with a relatively low level of skill and training in medical technology, such as a police officer, a fireman, or any other adult. It would be particularly desirable to have a handheld device into which a specimen could be introduced, after which the entire process would proceed automatically.

### SUMMARY

The foregoing and other advantages are achieved in accordance with the present disclosure which relates to a testing device that has a time trigger. The trigger is preferably made of a material which expands substantially upon absorbing specimen fluid, and it is mounted and positioned so as to contact and move another component of the device upon expanding through the absorption of specimen fluid.

Preferably, the trigger is mounted to the other component and is positioned to press against a stationary surface of the device upon expanding, so that the trigger causes the other component to move.

Preferably, the other component contains a surface coupled to receive specimen fluid from the specimen fluid receiver and has an area which contains a substance which interacts with the specimen fluid. The trigger is coupled to receive specimen fluid from the specimen fluid receiver in such a manner that there is a predetermined delay before the trigger expands sufficiently to move the other component, the specimen fluid interacting with the substance during the delay.

Preferably, a second component of the device is positioned to be contacted by the other component is constructed to absorb from the other component specimen fluid which has interacted with the substance. The second component may include an area containing a second substance, where interaction of specimen fluid with the second substance occurs automatically subsequent to the delay.

An assay device for a specimen solution, in one embodiment, comprises a receiver for receiving a specimen solution, a test member for testing the specimen solution, and a fluid actuated trigger capable of absorbing the specimen solution, another solution or a vapor and dimensionally expanding into an expanded state as it absorbs the specimen solution, the another solution or the vapor. The trigger in the expanded state causes the specimen solution to be tested by the test member.

An assay device for a specimen solution, in another embodiment, comprises a receiver for receiving a specimen solution, a test member for testing the specimen solution, and a fluid actuated time indicator capable of absorbing the specimen solution and dimensionally expanding into an expanded state as it absorbs the specimen solution. The time indicator indicates that testing of the specimen solution is complete.

### BRIEF DESCRIPTION OF THE DRAWINGS

The foregoing brief description and further objects, features and advantages of the present disclosure will be understood more completely from the following detailed description of presently preferred, but nonetheless illustrative, embodiments in accordance with the present disclosure, with reference being had to the accompanying drawings in which:

FIGS. 1A and 1B are perspective views of a fluid actuated trigger embodying the present disclosure, with FIG. 1A showing the trigger in an non-expanded state and FIG. 1B showing the trigger in its fully expanded state, after having been saturated with a liquid, or moist vapor;

FIG. 2 is a perspective view of a first embodiment of a device for performing biological assays in accordance with the present disclosure;

FIG. 3 is a partially cut-away perspective view of the device of FIG. 2 showing the internal construction;

FIGS. 4A and 4B are schematic representations of the internal construction of the device of FIG. 3, useful in describing the operation of the device, with FIG. 4A showing the device prior to the absorption of sample liquid by an internal trigger and FIG. 4B showing the device after absorption of the liquid;

FIG. 5 is a partially cut away perspective view of a second embodiment of an assay device in accordance with the present disclosure;

FIGS. 6A, 6B and 6C are schematic representations of the internal construction of the device of FIG. 5, useful in describing the operation thereof, with FIG. 6A showing the device prior to the absorption of sample liquid by an internal trigger, FIG. 6B showing the device after absorption of the liquid, and FIG. 6C showing the device a predetermined time after the absorption of liquid has started;

FIGS. 7A and 7B are schematic representations of the internal construction of an alternate embodiment of the test device, useful in describing the operation of the device, with FIG. 7A showing the device prior to the absorption of sample liquid by an internal trigger and FIG. 7B showing the device after absorption of the liquid;

FIGS. 8A and 8B are schematic representations of another embodiment of an assay device in accordance with the present disclosure, with FIG. 8A showing the device prior to the absorption of sample liquid by an internal trigger and FIG. 8B showing the device after absorption of the liquid;

FIG. 9A is a partial perspective view showing the forward portion of an assay device which is a secure sample collector embodying the present disclosure;

FIGS. 9B and 9C are schematic representations of the internal construction of the collector of FIG. 9A, with FIGS. 9A and 9B showing the collector prior to and subsequent to the absorption of liquid by an internal trigger;

FIG. 10A is a partial perspective view of the forward portion of an ultimate embodiment of a secure collector in accordance with the present disclosure;

FIGS. 10B and 10C are schematic representations of the internal construction of the collector useful in describing its operation;

FIG. 11A is a perspective view of a further embodiment of the device for performing biological assays;

FIG. 11B is a partially cut away perspective view of the device of FIG. 11A showing its internal construction;

FIGS. 11C and 11D are sectional views of the internal construction of the device of FIG. 11A showing its operation;

FIG. 12A is a perspective view of another embodiment of the device for performing biological assays;

FIG. 12B is a partially cut away perspective view of the device of FIG. 12A showing its internal construction;

FIG. 13A is a perspective view of a further embodiment of the device for performing biological assays;

FIG. 13B is a partially cut away perspective view of the device of FIG. 13A showing its internal construction;

FIG. 14A is a perspective view of still a further embodiment of the device for performing biological assays; and

FIGS. 14B and 14C are sectional views of the internal construction of the device of FIG. 14A showing its operation.

### DETAILED DESCRIPTION

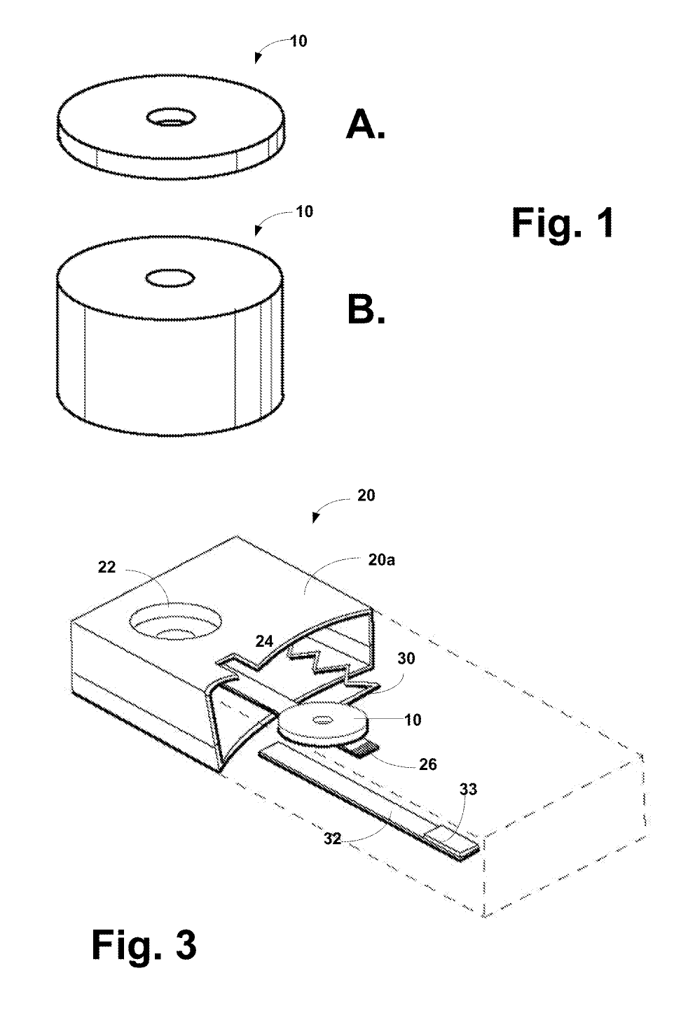

Turning now to the drawings, FIG. 1A is a perspective view of a fluid actuated trigger 10 embodying the present disclosure. Trigger 10 is preferably in the form of a disc made of compressed cellulose, or some other material that expands substantially in volume when it absorbs or is saturated with a liquid, usually aqueous in nature. Some liquids, such as alcohol, may not operate to expand cellulose material, but any material that can be expanded by any liquid may be used. While FIG. 1A illustrates trigger 10 in its initial state, FIG. 1B illustrates the trigger in its expanded state, after having absorbed a liquid, or the like.

One compressed disk 0.1 to 0.2 mm expands to 1.5 mm. Multiple disks add force and length. Force is uni-dimensional. A preferred material for use in practice of the present disclosure is the compressed cellulose material is manufactured by Blue Green Ind., Corp. with the following specifications:
Cellulose Sponge, Compressed,
100% Hydrocellulose (regenerated cellulose)
No additives
Color: White
Tear Strength: 8-10 lb (1×¼ inch section wet)
Pore size: 30-50 Durometer (Shore A Compressed Dry)
Elongation: 2% (Wet)
Compression Set: 10 to 1 (Dry)
Heat Resistance: 280 degrees F. continuous
Water Absorption: 15-17 times by weight (from dry state)
Density: 1.3-2.4 lb/ft3
Visual: Middle hole should be centered

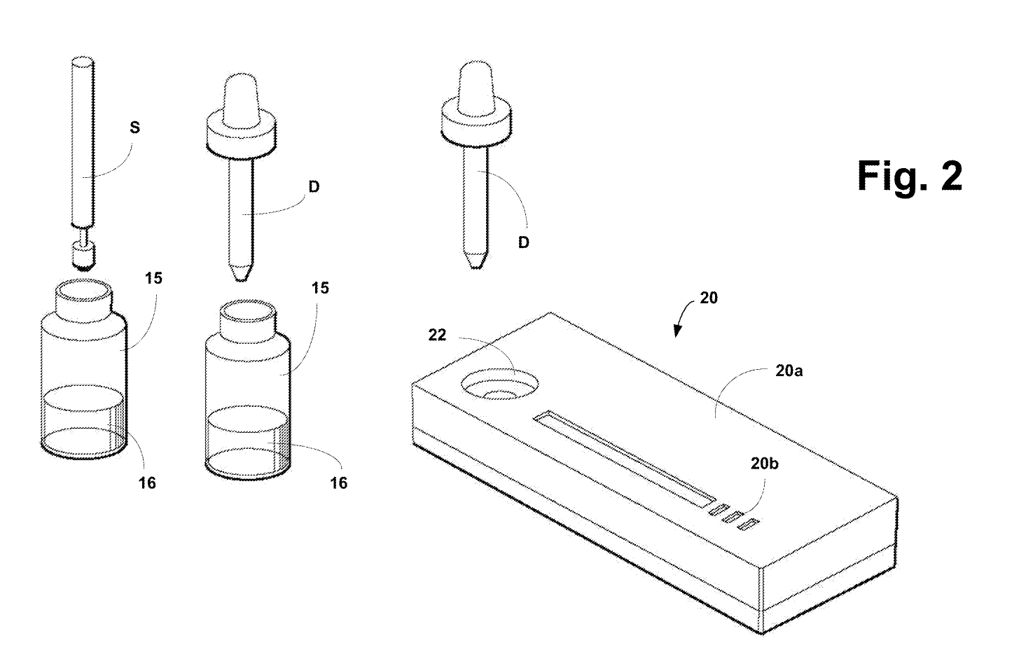

In accordance with one aspect of the present disclosure, a fluid actuated trigger is utilized to impart movement to components of an assay device. For example, FIG. 2 is a perspective view of a preferred device 20 for performing a biological assay. Initially, a biological specimen is taken with a sampler S and introduced into a container 15 containing a running buffer 16. Using a dropper D, or the like, the buffered specimen is introduced to an inlet well 22 of device 20.

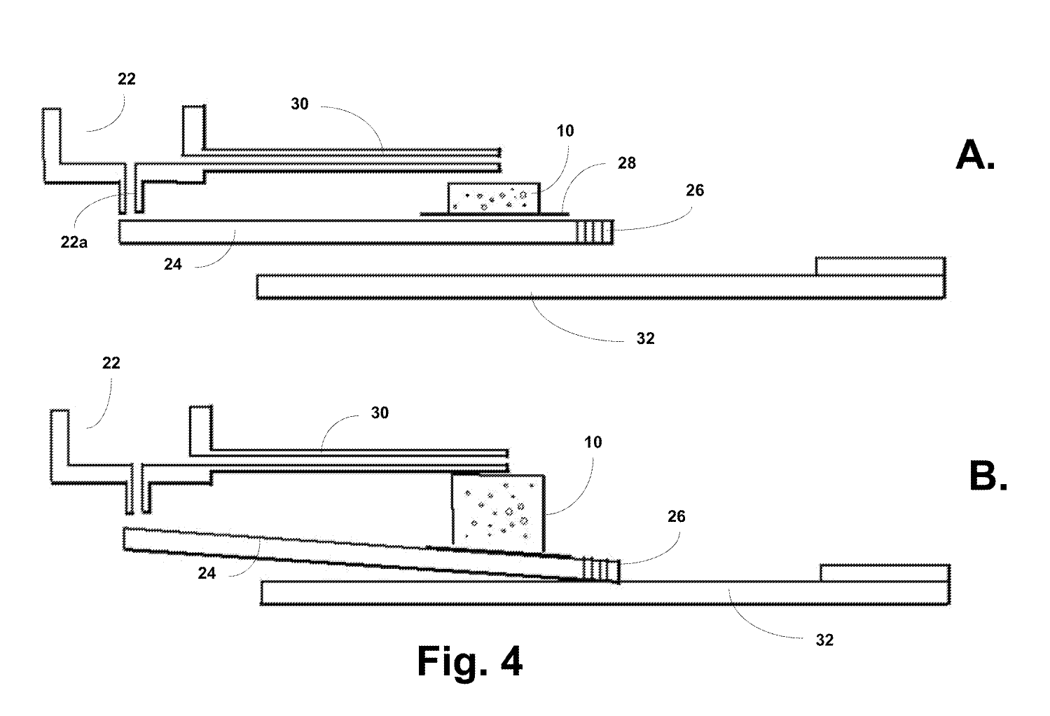

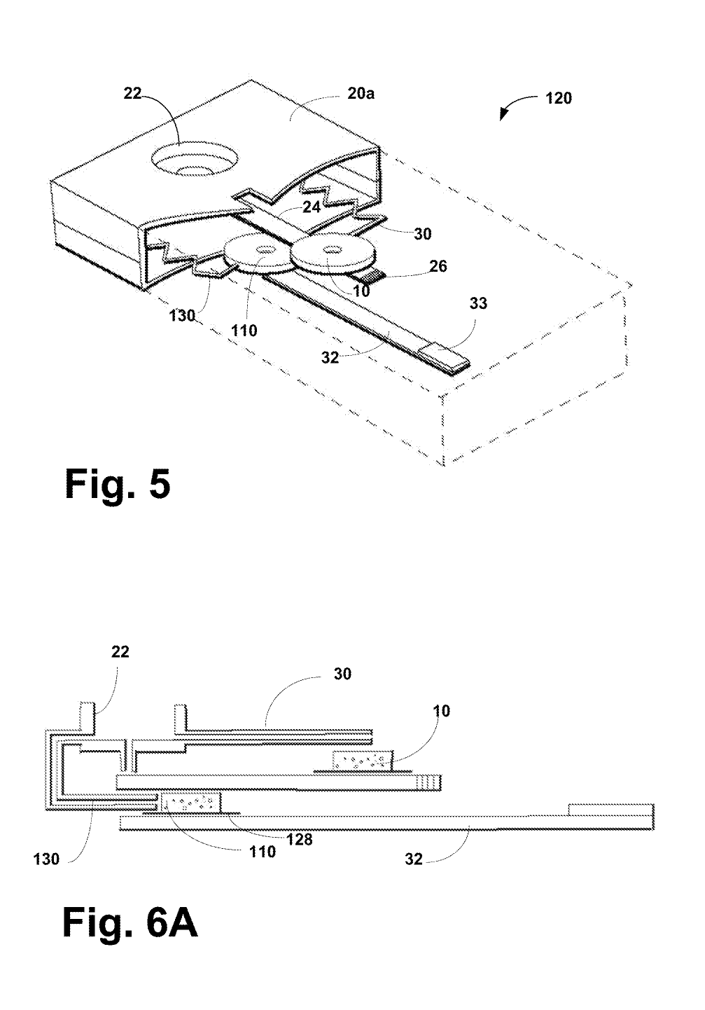

FIG. 3 is a partially cut away perspective view of the device 20 showing internal construction, and FIGS. 4A and 4B are schematic representations of that construction useful in describing the operation of device 20. The solution within the well 22 is dispensed via a capillary outlet 22a onto a sample pad 24 containing a treatment material, for example, a gold conjugate 26. Solution on pad 24 eventually reaches gold 26 and begins to incubate with the gold, in time producing an incubated liquid. A fluid actuated trigger 10 is mounted on pad 24, with a barrier 28 interposed between them that is impermeable to liquid from pad 24. Preferably, barrier 28 is a section of double-sided tape, also utilized to retain trigger 10 in position.

A capillary tube 30 is connected between the well 22 and trigger 10, allowing liquid from well 22 to be introduced gradually to trigger 10. As trigger 10 absorbs liquid from well 22, it begins to swell, bearing upon the stationary undersurface of the top wall 20a of device 20 and forcing pad 24 to bend downward, as illustrated in FIG. 4B. A test strip 32 preferably made of nitrocellulose is mounted at a fixed position below pad 24 and eventually pad 24 bends sufficiently to come into contact with test strip 32, distributing the incubated solution to it. Typically, test strip 32 would be treated with a reagent 33 intended to react with the incubated solution on pad 24. The reagent may for example change color to indicate the results of a test. The treated area 33 may be observed through a window 20b in device 20, as shown in FIG. 2. By design, the dimensions of capillary tube 30 and the saturation time of trigger 10 are calculated to permit complete incubation on pad 24 before it comes into contact with strip 32.

Those skilled in the art will appreciate that, through the use of trigger 10 as disclosed, it becomes possible to perform automatically a two step operation with a programmed delay between the steps. This eliminates the inconsistency and errors that can be introduced when those steps are performed manually by an operator. It also makes it possible for the entire test to be performed successfully by a relatively unskilled operator.

The proper width, size and shape of the various channels within the apparatus can be determined via empirical measurements. Thus, if the expansion occurs to quickly to allow for the proper reaction time, for example, one can simply diminish the size of the channel that provides liquid to the trigger for expansion.

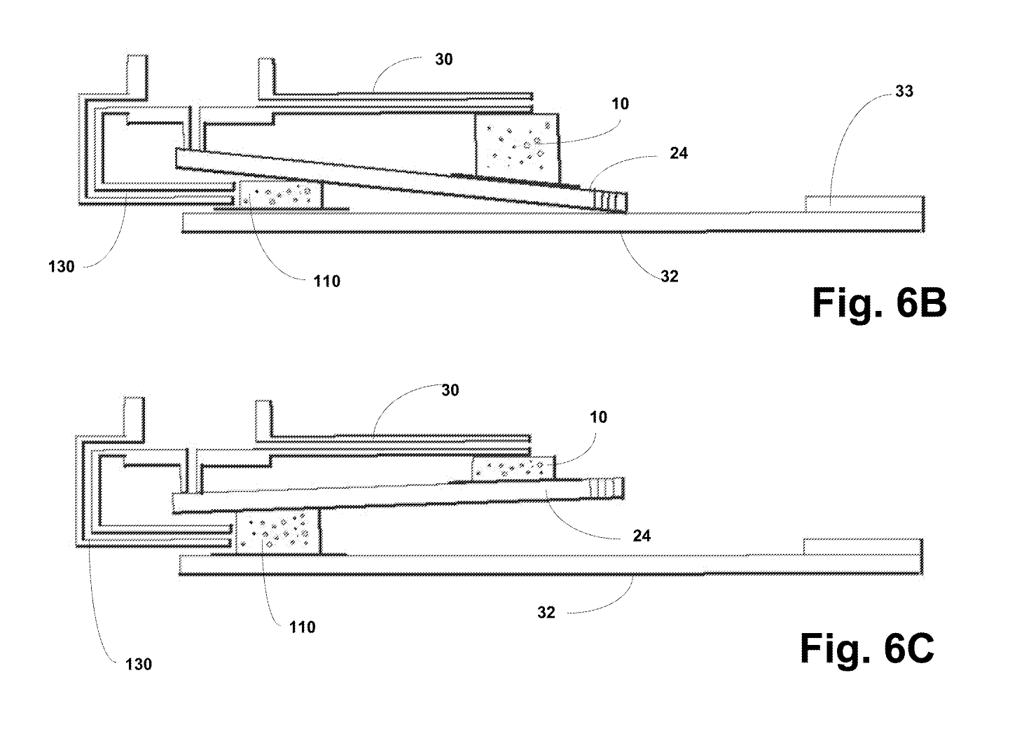

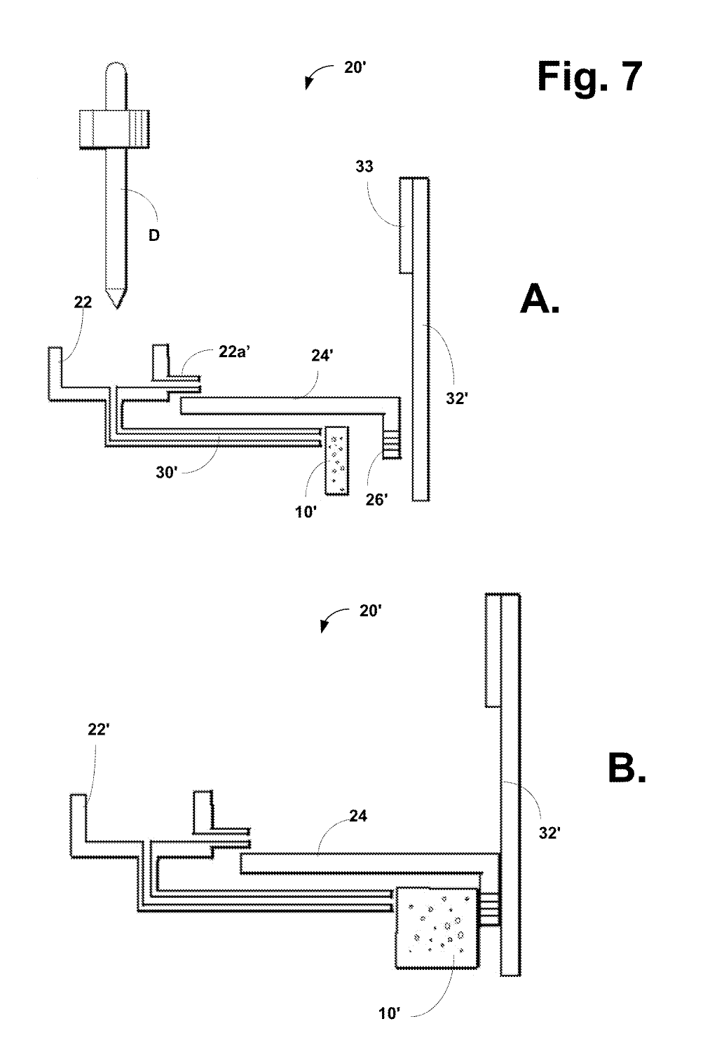

It should be appreciated that, by adding additional liquid actuated triggers, it would be possible to have additional steps performed in a testing device, all with their own timing. For example, FIG. 5 is a partially cut away perspective view of a second embodiment 120 of a testing device in accordance with the present disclosure. In part, device 120 is identical to device 20, and the identical elements are represented by the same reference characters as in device 20. The primary difference is that device 120 includes a second liquid actuated trigger 110, which is connected to well 22 through a capillary tube 130. FIGS. 6A, 6B and 6C are schematic representations of the internal construction of device 120, useful in describing the operation thereof.

To the extent illustrated in FIGS. 6A and 6B, the operation of device 120 whereby a test indication is provided in treated area 33 is identical to that of device 20. The description already provided with respect to FIGS. 4A and 4B is equally applicable and will not be repeated here. The second liquid actuated trigger 110 is mounted on test strip 32 by means of a second double-sided tape, or the like (ex. friction pins), 128 which holds it in position and also acts as an impermeable barrier. A sample introduced into well 22 will be introduced to trigger 110 through tube 130. As a result, trigger 110 will begin to swell. At a time determined by the construction of tube 130, trigger 110 will have swelled enough to cause separation of pad 24 and strip 32, at which point incubated solution is no longer provided to strip 32. By design, tube 130 will be constructed so that trigger 110 will not act for a sufficient time to permit strip 32 to perform its test. However, tube 130 will also be of such a construction as to assure that pad 24 and strip 32 will be separated after a predetermined time. This will assure that too much incubated solution is not provided to strip 32. For some reactions, providing too much incubated fluid could cause inaccuracies or be detrimental to the reaction taking place on strip 32. Thus, test device 120 assures that there is sufficient incubation on pad 24 before it comes into contact with strip 32, that contact between pad 24 and strip 32 is for a sufficient time to provide an adequate amount of incubated solution, and that the contact is not for such a long time as to provide too much incubated solution. At the same time, the operation of test device is entirely automatic once well 22 is filled and does not require skill or diligence on the part of the operator.

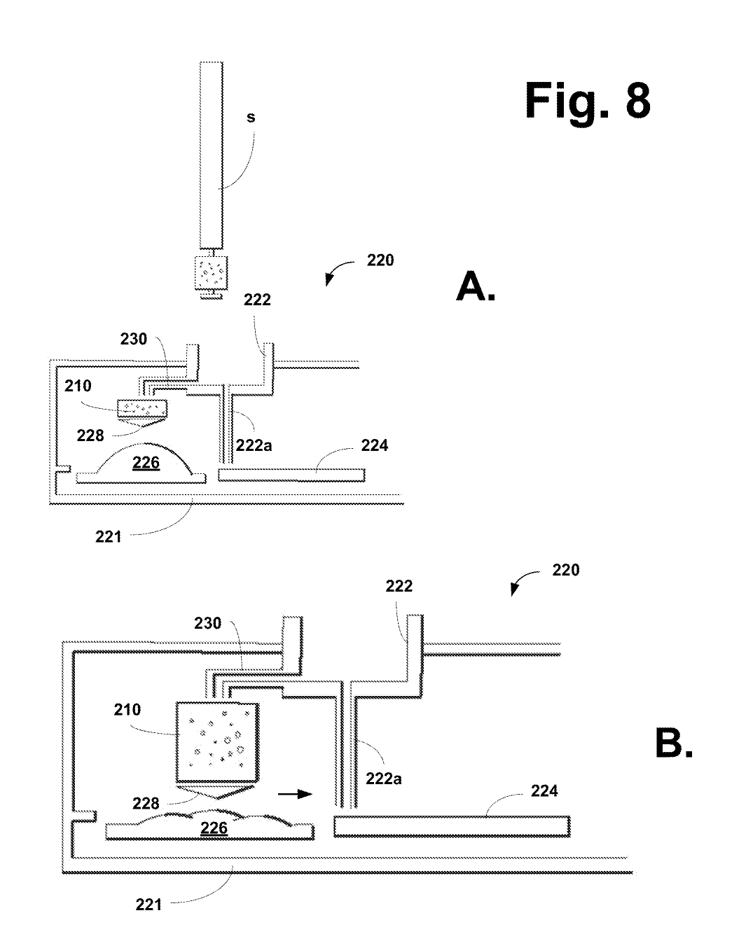

FIGS. 7A and 7B are schematic representations of an alternate embodiment 20′ of test device 20. In this embodiment, well 22 is coupled to a liquid actuated trigger 10′ through a capillary tube 30′. A pad 24′ with gold 26′ thereon is mounted for lateral movement, either with or against gravity, and a test strip 32′ is positioned vertically at a lateral distance from pad 24′. A solution to be tested is provided to well 22, for example with a dropper D, and is deposited upon pad 24′ through an outlet 22a′. Solution applied to pad 24′ will cooperate with gold 26′ to produce an incubated solution. Fluid supplied through tube 30′ causes trigger 10′ to swell and, in time, it will contact on pad 24′, forcing it to the right, into contact with strip 32′. This will cause incubated solution to be applied to strip 32′, and a predetermined test will be performed on the strip, with treated portion 33 ultimately showing the intended test result. As was the case with tube 30, tube 30′ is designed to assure a sufficient incubation time on pad 24′ before pad 24 touches strip 32′.

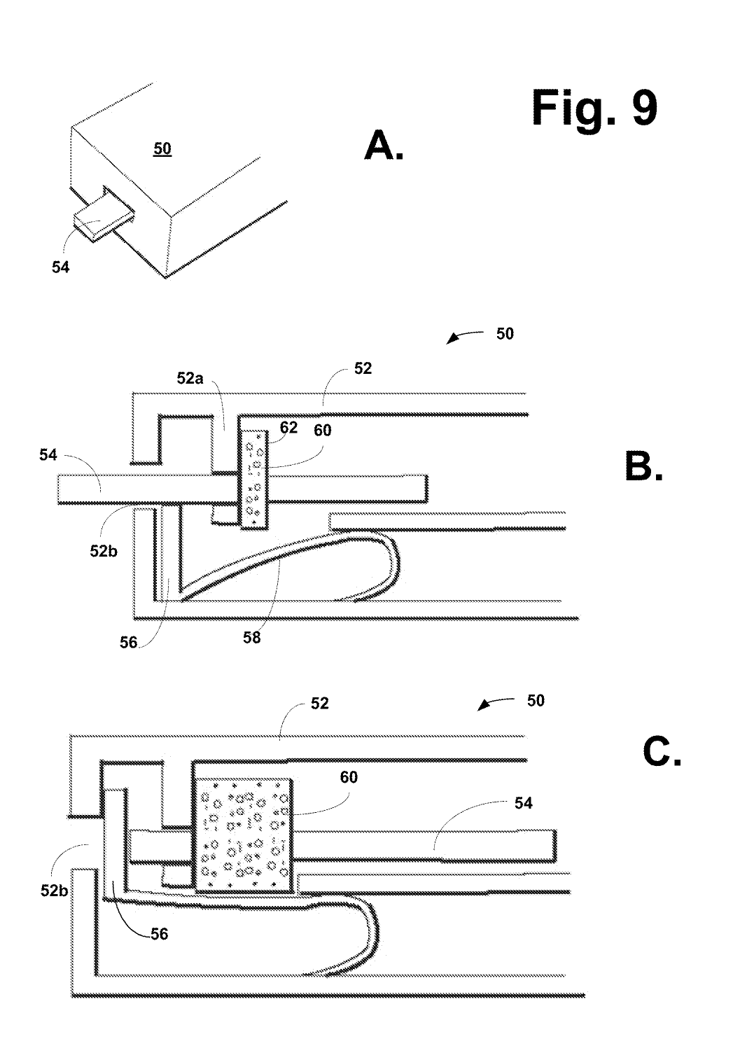

FIGS. 8A and 8B are schematic representations of another embodiment 220 of an assay device in accordance with the present disclosure. Device 220 includes a hollow body 221 and a well 222. A specimen liquid to be tested may be introduced to well 222, for example with a sample S. Within body 221, there is provided a test strip 224 which, will typically include an indicating portion (not shown) reflecting the result of the assay. Also within body 221, there is provided a package 226 containing a reagent to be applied to strip 224. Positioned above package 226 is a liquid actuated trigger 210, to the bottom of which is attached at element 228, for example a piercing element, to open package 226. A solution introduced to well 222 is introduced onto test strip 224 through outlet 222a. At the same time, liquid is also introduced to trigger 210 through a capillary tube 230 and begins swelling trigger 210. At the same time, test strip 224 is adequately loaded with a specimen liquid. At a time determined by the construction of capillary tube 230, element 228 is forced into package 226, breaking it open and allowing the reagent therein to leak upon test strip 224 as indicated by the arrow. This reagent is then absorbed by the test strip, allowing the intended test to take place.

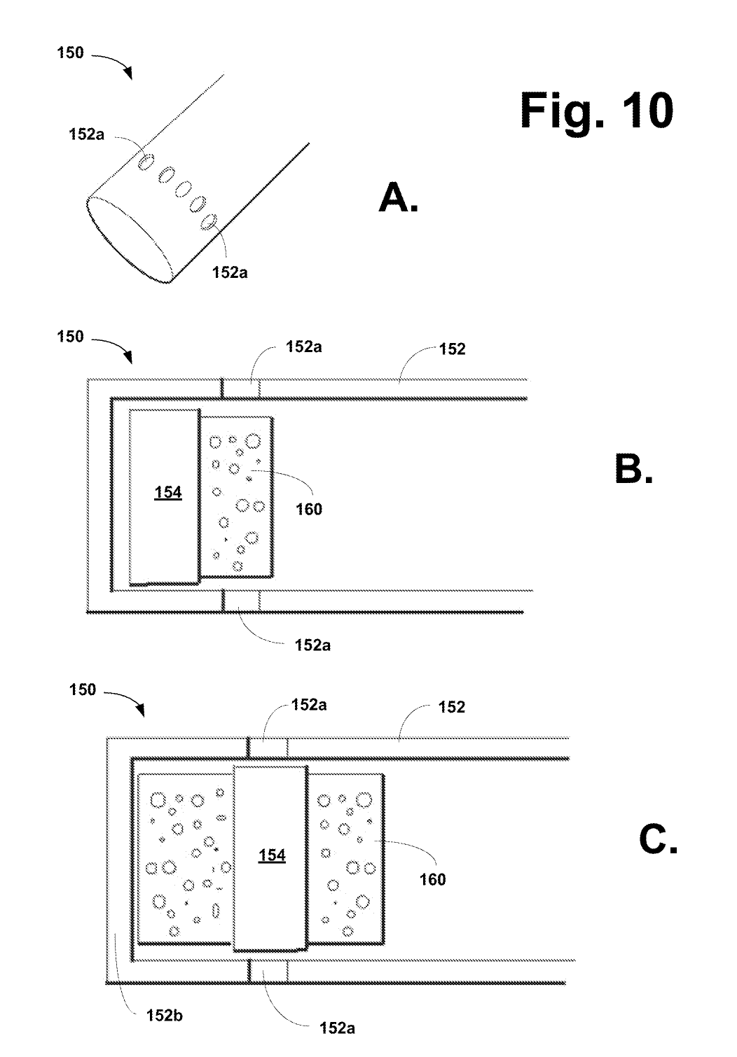

In addition to providing an automatic fluid testing device, a liquid trigger can provide a secure specimen collecting device. For example, FIG. 9A is a partial perspective view showing the forward portion of a secure sample collector 50 embodying the present disclosure. Collector 50 includes an enclosure or body 52 from which a sample pad 54 protrudes. Collector 50 may be used to collect saliva samples by placing pad 54 on the tongue and saturating it with saliva. Collector 50 is a secure collector, in that, once pad 54 is saturated, it will be withdrawn into the enclosure 52, protecting it against damage and contamination.

FIGS. 9B and 9C are schematic representations of the internal construction of collector 50. Strip 54 protrudes forwardly out of the enclosure 52 through a window 52b. In addition, enclosure 52 contains an internal upright stationary wall 52a and pad 54 protrudes through an opening in that wall and moves freely therein. To the rear of wall 52a, a liquid actuated trigger 60 is mounted on pad 54 so that its rear portion 52 is secured to the pad. Forward of portion 62, however, trigger 60 may move freely over pad 54. An upright door 56 is mounted within enclosure 52 by means of a resilient loop 58 which urges it upward. However, with pad 54 in its pre-use position, door 56 is retained in a downward position (FIG. 9B) below pad 54.

When pad 54 is placed in a patient's mouth to take a saliva sample, the pad begins to absorb liquid, and that liquid is transferred to trigger 60. Trigger 60 begins to expand, with its forward face bearing on wall 52a and since the rear portion 62 is secured to pad 54, pad 54 is drawn rearward into an enclosure 52 through the expansion of trigger 60 (FIG. 9C). When the front of pad 54 passes rearward of door 56, the resilience of loop 58 forces door 56 upward, closing off the window 52b and protecting pad 54 in a sealed compartment.

It will be appreciated that device 50 is not only a secure collecting device, but it would also make it possible to perform tests inside it, without the risk that internal reagents might find there way onto pad 54 and into the patient's mouth. For example, the rear portion of device 50 could include structure such as shown in FIG. 8A to apply a reagent to pad 54 after door 56 is closed.

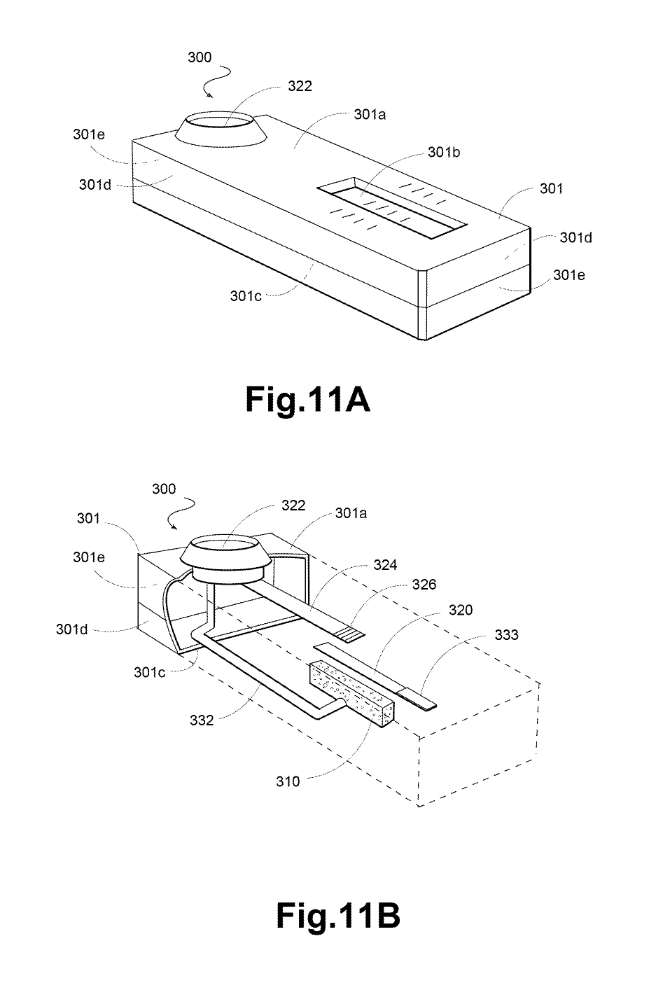

FIG. 10A is a partial perspective view of the forward portion of an alternate embodiment 150 of a secure collector in accordance with the present disclosure. FIGS. 10B and 10C are schematic representations of the internal construction of collector 150 useful in describing its operation. Collector 150 has a generally cylindrical enclosure 152 containing an array of sampling ports 152a providing access to the interior of the enclosure 152. A generally cylindrical sleeve 154 is mounted within enclosure 152 for longitudinal sliding movement. Mounted inside sleeve 154 is a compressed cellulose plug (trigger) 160 which is secured to the rear of sleeve 154. The forward portion of cellulose plug 160 extends freely into the interior sleeve 154.

In operation, a saliva sample may be taken by placing the forward end of collector 150 into the mouth and saturating it with the tongue. Saliva then seeps through the ports 152a, into the cellulose plug 160. As plug 160 absorbs liquid, it begins to expand, and its forward portion bears against the forward wall 152b of enclosure 152, forcing sleeve 154 rearward. Eventually, sleeve 154 reaches the position shown in FIG. 10C, where it blocks the ports 152a, and no further liquid can be absorbed. In addition, collected saliva remains in the cellulose plug 160, protected by the enclosure 152. As was the case with device 50, the right hand portion of device 150 could include structure such as that shown in FIG. 8A to apply a reagent to plug 160 after sleeve 154 blocks ports 152a.

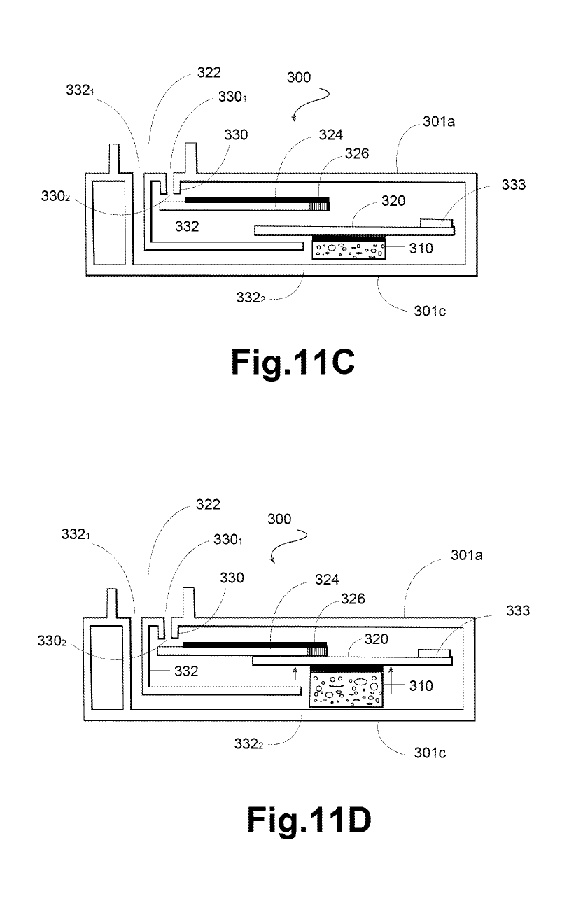

FIGS. 11A-11D collectively illustrate a further embodiment of the device for performing biological assays, denoted by reference numeral 300, where FIG. 11A is a perspective view of the device 300, FIG. 11B is a partially cut away perspective view of the device 300 showing its internal construction, and FIGS. 11C and 11D are sectional views of the internal construction of the device 300 showing its operation. The device 300 comprises a housing 301 that can include a top wall 301a, a bottom wall 301c, side walls 301d, and end walls 301e. Disposed within the housing 301 is a first capillary tube 330, a second capillary tube 332, an elongated sample pad 324, an elongated test strip 320, and a fluid actuated trigger 310. The structure and operation of the sample pad 324, test strip 320, and fluid actuated trigger 310 are substantially identical to the sample pads, test strips and fluid actuated triggers described previously, except where noted below.

Referring still to FIGS. 11A-11D, the top wall 301a of the housing 301 includes a viewing window 301b and a fluid specimen inlet well 322. The first capillary tube 330 has a first opening 330₁ that communicates with the specimen inlet well 322 and a second opening 330₂ that communicates with a first end 324₁ of the sample pad 324. The second capillary tube 332 has a first opening 332₁ that communicates with the specimen inlet well 322 and a second end 332₂ that communicates with the trigger 310. The elongated sample pad 324 is fixedly disposed within the housing 301. The sample pad 324 can be made of, for example, glass fiber, cotton linter, or polyester. A treatment material 326 can be disposed on the sample pad 324, for example, at a second end 324₂ thereof or at an other suitable location of the sample pad 324. In other embodiments, the additional treatment materials of the same or different type may be disposed on the sample pad 324. In one exemplary embodiment, the treatment material 326 can comprise a gold conjugate. In other embodiments, the treatment material 326 can comprise, without limitation, a horse raddish peroxidase conjugated antibody, an alkaline phosphatase conjugated antibody, a selenium conjugated antibody, a silver conjugated antibody, a colored latex conjugated antibody, a charcoal/carbon conjugated antibody, or an isotope conjugated antibody. In still other embodiments, the treatment material 326 can comprise a colored or uncolored glass particle, conjugated antigens, conjugated protein A, conjugated protein G, conjugated peptides, conjugated genetic markers, or a conjugated Fluorescein isothiocyanate (FITC), which produces an assay that is fluorescent in nature requiring a fluormeter type reader. In further embodiments, biotin, avidin, or streptavidin may be used as the treatment material 326 to attach and enhance sensitivity. In still further embodiments, polymerase chain reaction (PCR) technology for DNA/RNA may be used as the treatment material 326. The elongated test strip 320 is disposed below the sample pad 324 in the housing 301 and is fixedly attached to the top surface of the trigger 310 by a fluid impermeable barrier 328 (e.g. a section of double-sided tape). The bottom surface of the trigger can engage or be suspended above the bottom wall 301c of the housing 301. The elongated test strip 320 can be made of nitrocellulose or any other material suitable for chemical and biological testing. The test strip 320 can include one or more treated areas 333. Each of the one or more treated areas 333 of the test strip 320 may be treated with a reagent intended to react with an incubated specimen solution. The reagent(s) may for example change color to indicate the results of a test.

In operation, a specimen solution introduced into the specimen inlet well 322 is concurrently dispensed via the first and second capillary tubes 330, 332 onto the sample pad 324 and the trigger 310. More specifically, the first capillary tube 330 dispenses the specimen solution onto the elongated sample pad 324 and the second capillary tube 332 dispenses the specimen solution onto the trigger 310.

The specimen solution dispensed onto the sample pad 324 by the first capillary tube 330 may be dispensed at the first end 324₁ of the sample pad 324. The specimen solution then travels down the sample pad 324 to be incubated by the treatment material 326. The specimen solution is incubated by the treatment material 326 for a predetermined time period to produce a completely incubated solution.

The specimen solution dispensed via the second capillary tube 332 onto the trigger 310 is absorbed by the trigger 310. As the trigger 310 absorbs the specimen solution, it expands in height and raises the test strip 320 toward the top wall 301a of the housing 301 as illustrated in FIG. 11D until the test strip 320 contacts the area or areas of the sample pad 324 where the treatment material 326 has incubated the specimen solution and produced a completely incubated solution. The trigger 310 never becomes part of or contacts the reagent(s) or the sample pad 310. As explained earlier, the predetermined time period for complete incubation is equal to the time it takes for the specimen solution to flow through the second capillary tube 332 and expand the trigger 310. Accordingly, the dimensions of the capillary tubes 330, 332 and the expansion time of trigger 310 are calculated to permit complete incubation of the specimen solution on the sample pad 324 before the test strip 320 comes into contact with it.

When the test strip 320 contacts the sample pad 324, the incubated specimen solution produced on the sample pad 324 is absorbed by the test strip 320. The treated area 333 of the test strip 320 reacts with the incubated specimen solution and, for example, changes color to indicate the results of a test. The treated area 333 of the test strip 320, which has been raised within the housing 301 so that it is adjacent the top wall 301a thereof, may be viewed through the window 301b in the housing top wall 301a, as shown in FIG. 11A. Raising the test strip 320 so that it is adjacent to the top wall 301a of the housing 301 reduces the depth of field. The reduced depth of field allows a reader, such as a CCD camera, an optical scanner, a densitometer, and the like, to read the treated area(s) 333 of the test strip 320 with more accuracy.

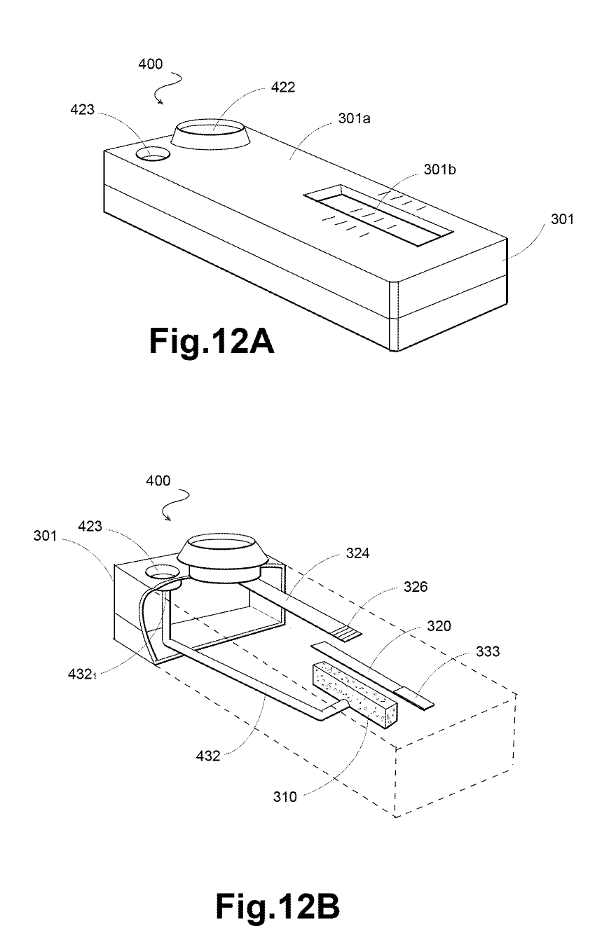

FIGS. 12A and 12B collectively illustrate another embodiment of the device for performing biological assays, denoted by reference numeral 400, where FIG. 12A is a perspective view of the device 300 and FIG. 12B is a partially cut away perspective view of the device 400 showing its internal construction. The device 400 is substantially identical in structure and operation to the device 300 embodied in FIGS. 11A-11D, except the first opening 432₁ of the second capillary tube fluid communicates with a second fluid inlet well 423 provided in the top wall 301a of the housing 301 instead of the fluid specimen inlet well 422. The specimen solution or any other suitable solution or vapor capable of expanding the trigger 310 can be introduced into the second fluid inlet well 423 for use in activating the trigger 310 for incubation timing.

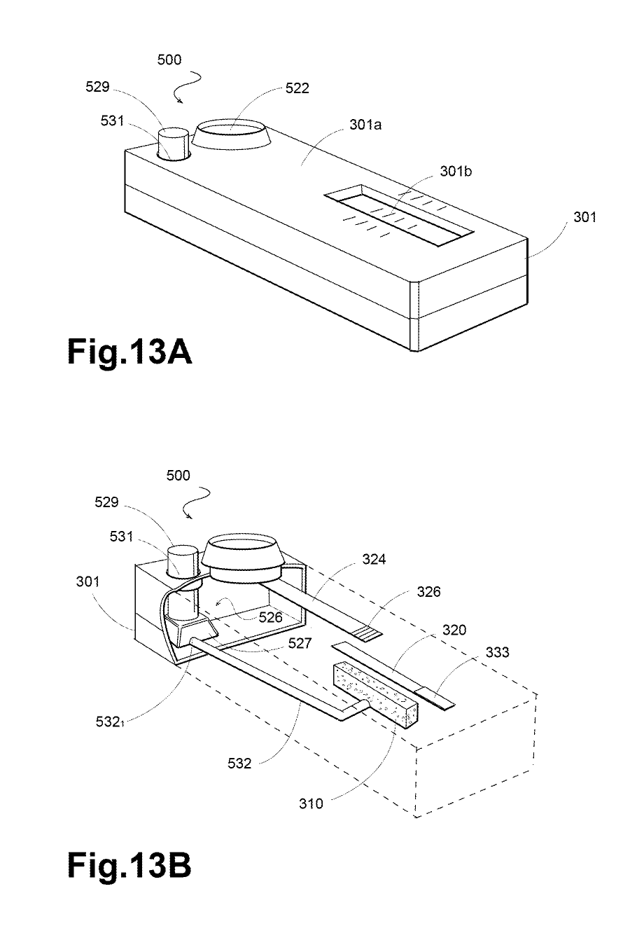

FIGS. 13A and 13B collectively illustrate still another embodiment of the device for performing biological assays, denoted by reference numeral 500, where FIG. 13A is a perspective view of the device 500 and FIG. 13B is a partially cut away perspective view of the device 500 showing its internal construction. The device 500 is substantially identical in structure and operation to the device 300 embodied in FIGS. 11A-11D, except instead of communicating with the fluid specimen inlet well 522, the first opening 532₁ of the second capillary tube 532 fluid communicates with a finger actuated fluid pump 526. The pump 526 can include a solution- or vapor-filled reservoir 527 disposed in the housing 301 and a trigger button 529 that extends out of the housing 300 through an aperture 531 in the top wall 301a thereof. When the trigger button 529 of the pump 526 is pressed down into the housing 300, it forces the solution or vapor out of the reservoir 527 and into the first opening 532₁ of second capillary tube 532. The second capillary tube 532 then dispenses the solution or vapor onto the trigger 310 as described above with respect to the device 300 of FIGS. 11A-11D.

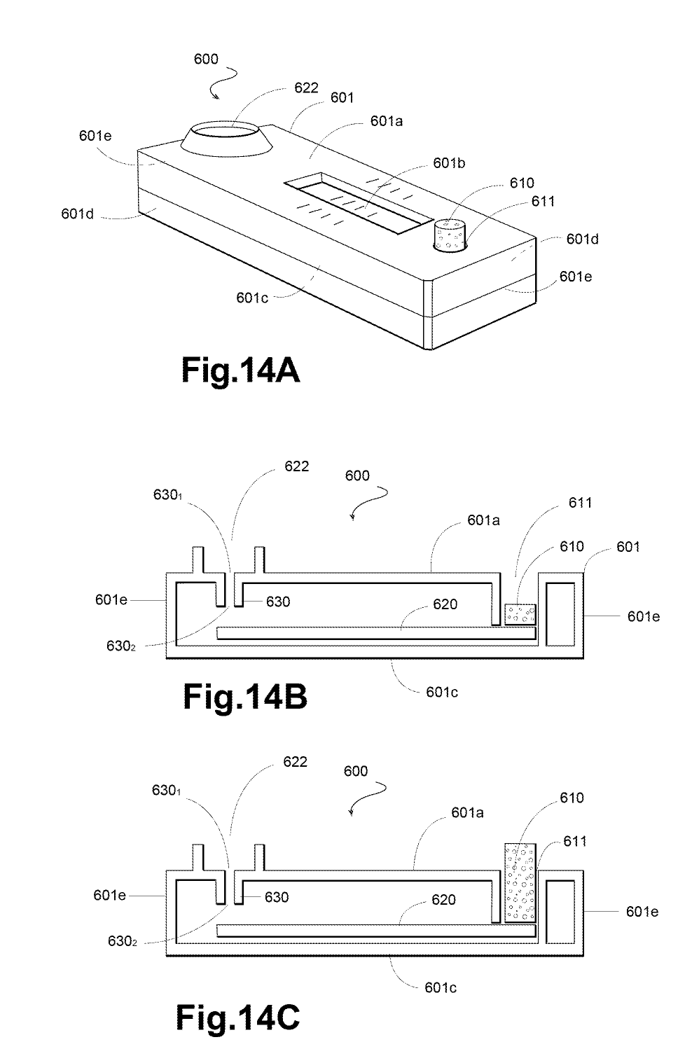

FIGS. 14A-14C collectively illustrate still a further embodiment of the device for performing biological assays, denoted by reference numeral 600, where FIG. 14A is a perspective view of the device 600 and FIGS. 14B and 14D are sectional views of the internal construction of the device 600 showing its operation. The device 600 comprises a housing 601 that can include a top wall 601a, a bottom wall 601c, side walls 601d, and end walls 601e. Disposed within the housing 601 is a capillary tube 630, an elongated test strip 620, and a fluid actuated time indicator 610. The test strip 620 is similar to the test strips described previously. The top wall 601a of the housing 601 includes a viewing window 601b, a bore 611 for containing the time indicator 610, and a fluid specimen inlet well 622. The capillary tube 630 has a first opening 630₁ that communicates with the specimen inlet well 622 and a second opening 630₂ that dispenses a specimen solution onto the test strip 620. The bottom of the bore 611 communicates with the interior of the housing 601. The elongated test strip 620 can be made of nitrocellulose or any other material suitable for chemical and biological testing. The test strip 620 can include one or more treated areas 633. Each of the one or more treated areas 633 of the test strip may be treated with a reagent intended to react with a specimen solution. The reagent(s) may for example change color to indicate the results of a test. The indicator 610 can be made of the same material as the fluid activated trigger describe above, i.e., a material which expands substantially upon absorbing a fluid specimen. For example, the time indicator 610 can be a disc made of compressed cellulose, or some other material that expands substantially in volume when it absorbs or is saturated with a liquid or vapor.

In operation, a specimen solution introduced into the fluid specimen inlet well 622 is dispensed via the capillary tube 630 onto the test strip 620. The treated area(s) 633 of the test strip 620 reacts with the specimen solution and, for example, changes color to indicate the results of a test. Some of the specimen solution also travels to the indicator 610 and is absorbed thereby. As the time indicator 610 absorbs the specimen solution, it expands in height within the bore 611. When the time indicator 610 emerges from the bore 611 (FIG. 14C) it indicates that the test performed by the test strip 620 has been completed. The treated area 633 of the test strip 620 may be viewed through the window 601b in the top wall of the housing 601, as shown in FIG. 14A. A reader, such as a CCD camera, an optical scanner, a densitometer, and the like may be used for reading the treated area(s) 633 of the test strip 620.

While the above describes the preferred embodiments, various other modifications and additions will be apparent to those of skill in the art. Such variations are intended to be covered by the following claims.

What is claimed is:

1. An assay device for a specimen solution, the assay device comprising:
a receiver for receiving a specimen solution;
a test member for testing the specimen solution; and
a fluid actuated trigger capable of absorbing the specimen solution, another solution or a vapor and dimensionally expanding into an expanded state as it absorbs the specimen solution, the another solution or the vapor;
wherein the trigger in the expanded state causes the specimen solution to be tested by the test member.

2. The assay device of claim 1, wherein the trigger in the expanded state causes the specimen solution to be tested by the test member by moving the test member within the device as it expands into the expanded state.

3. The assay device of claim 2, further comprising a sample pad for incubating the specimen solution.

4. The assay device of claim 3, wherein the sample pad includes a substance for incubating the specimen solution.

5. The assay device of claim 4, further comprising a capillary tube for transferring the specimen solution from the receiver to the sample pad for incubation by the sample pad.

6. The assay device of claim 3, further comprising a capillary tube for transferring the specimen solution from the receiver to the sample pad for incubation by the sample pad.

7. The assay device of claim 3, wherein the test member contacts the sample pad when the test member is moved by the trigger.

8. The assay device of claim 7, further comprising a fluid path between the receiver and the trigger, the path for allowing the specimen solution to be incubated before the trigger reaches the expanded state.

9. The assay device of claim 8, wherein the fluid path is formed by a capillary tube.

10. The assay device of claim 2, further comprising a housing for enclosing the test member, the housing member having a portion through which the result of the test is visible, wherein trigger moves the test member toward the window.

11. The assay device of claim 1, wherein the test member includes an area treated with a reagent.

12. The assay device of claim 11, wherein the specimen solution reacts with the reagent to generate a test result.

13. The assay device of claim 1, further comprising a capillary tube for transferring the specimen solution from the receiver to the trigger for absorption by the trigger.

14. The assay device of claim 1, further comprising a second receiver for receiving the specimen solution, the another solution or the vapor.

15. The assay device of claim 14, further comprising a fluid path between the second receiver and the trigger, the path for allowing the specimen solution to be incubated before the trigger reaches the expanded state.

16. The assay device of claim 15, wherein the fluid path is formed by a capillary tube.

17. The assay device of claim 14, further comprising a capillary tube for transferring the specimen solution, the another solution or the vapor from the second receiver to the trigger for absorption by the trigger.

18. The assay device of claim 1, further comprising a pump for delivering a solution or a vapor to the trigger for absorption thereby.

19. The assay device of claim 18, further comprising a fluid path between the pump and the trigger, the path for allowing the specimen solution to be incubated before the trigger reaches the expanded state.

20. The assay device of claim 19, wherein the fluid path is formed by a capillary tube.

21. The assay device of claim 18, further comprising a capillary tube for transferring the solution or vapor from the pump to the trigger for absorption by the trigger.

22. An assay device for a specimen solution, the assay device comprising:
a receiver for receiving a specimen solution;
a test member for testing the specimen solution; and
a fluid actuated time indicator capable of absorbing the specimen solution and dimensionally expanding into an expanded state as it absorbs the specimen solution;
wherein the time indicator indicates that testing of the specimen solution is complete.

23. The assay device of claim 22, further comprising a capillary tube for transferring the specimen solution from the receiver to the time indicator for absorption by the time indicator.

24. The assay device of claim 22, further comprising a capillary tube for transferring the specimen solution from the receiver to the test member.

25. The assay device of claim 22, further comprising a housing for enclosing the test member, the housing including a bore for containing the time indicator.
